(19) United States
(12) Patent Application Publication
(10) Pub. No.: US 2010/0183781 A1
(43) Pub. Date: Jul. 22, 2010

(54) SYSTEM, METHOD AND APPARATUS FOR CONTROLLING MICROBIOLOGICAL CONTAMINATION IN COMMERCIAL FREEZE DRYERS USING REFLECTIVE ELECTROMAGNETIC ENERGY TECHNOLOGY

(75) Inventors: James L. Marsden, Manhattan, KS (US); Marie Moody, New Berlin, WI (US)

Correspondence Address: SHUTTS & BOWEN, LLP, 100 S. ASHLEY DRIVE, SUITE 1500, TAMPA, FL, 33602, US

(73) Assignee: Stella & Chewy's, LLC, Muskego, WI (US)

(21) Appl. No.: 12/690,664

(22) Filed: Jan. 20, 2010

Related U.S. Application Data
(60) Provisional application No. 61/145,822, filed on Jan. 20, 2009.
(60) Provisional application No. 61/145,835, filed on Jan. 20, 2009.

(51) Int. Cl. A23L 3/3409; A61L 2/20; A61L 2/26; B01J 7/00

(52) U.S. Cl. 426/236; 422/305; 422/186.04; 422/186.12; 422/292; 422/29; 426/320

(57) ABSTRACT

The system, method and apparatus involves the control of microbiological contamination of food processing equipment such as a commercial freeze dryer using oxidation gases. An oxidative gas generator such as a REME system produces low levels of ozone, vaporized hydrogen peroxide and other oxidative gases. These compounds act as anti-microbial agents and systematically inactivate bacteria, viruses, yeast and mold in the air and on surfaces around and inside the food processing equipment.

### CROSS-REFERENCE TO RELATED APPLICATIONS

This application relates to the same subject matter as co-pending provisional patent application Ser. No. 61/145,822, filed by the same applicant on Jan. 20, 2009, and co-pending provisional patent application Ser. No. 61/145,835, filed by the same applicant on Jan. 20, 2009. This application claims the Jan. 20, 2009 filing date as to the common subject matter.

### BACKGROUND OF THE INVENTION

1. Field of Invention

The present invention relates to a system, method and apparatus for the control of microbiological contamination. It applies particularly to control in commercial freeze dryers, and will be described with particular reference thereto.

2. Background of Invention

The current systems, methods and equipment for cleaning and sanitizing commercial freeze dryers, especially those used in the processing of food for humans and animals, involve the application of detergents and liquid sanitizers. This application may be done on a daily basis or less frequently.

These traditional methods and equipment do not involve the use of plasma, gas or vapor based anti-microbial compounds. When foam detergents and/or liquid sanitizers are used, some areas in an environment or on a surface of a particular piece of equipment may not be treated during the cleaning or sanitation process. The failure may be a function of ability of the product used to cover the entire surface. Also, the application is often performed by people, and human errors may be made in the application of the detergents and sanitizers. If the environment around, on or within a piece of equipment becomes contaminated during operations and is not properly treated afterwards, microorganisms may colonize inside the equipment and act as a vector of contamination during the freeze drying process. In some cases wherein cleaning and sanitizing occurs infrequently, the potential for recontamination or increased contamination may exist for long periods of time.

The present invention provides a system, method and apparatus using Reflective Electro-Magnetic Energy (REME) for enhanced control of microbiological contamination in equipment such as commercial freeze dryers.

Where the system of the invention is mounted external to the unit, the equipment may be cleaned and sanitized either through traditional means or by using a Clean-in-Place (CIP) system. The REME cell would act as an adjunct to routine cleaning and sanitation. The combination of proper cleaning and sanitation and intermittent and/or continuous treatment with the REME system will substantially reduce the risk of recontamination during the freeze drying process.

### SUMMARY OF THE INVENTION

In accordance with one aspect of the invention, a system, method and apparatus for the control of microbiological contamination is described herein. It applies particularly to control in commercial freeze dryers.

The system and method involve the decontamination of commercial freeze dryers using advanced oxidation gases produced by Reflective Electro-Magnetic Energy (REME) technology. The REME system produces into the external environment low levels of ozone, vaporized hydrogen peroxide and other oxidative gases. These compounds act as anti-microbial agents and systematically inactivate bacteria, viruses, yeast and mold in the air and on surfaces inside commercial freeze dryers.

The system, method and apparatus described above eliminate or reduce biological hazards, including microbiological pathogens such as those found in human and animal food processing equipment such as commercial freeze dryers. This helps prevent potential recontamination of food products, vitamins and pharmaceuticals during processes such as the freeze drying process.

This system, method and apparatus allow for the introduction of anti-microbial compounds in gas or vapor form which address all internal surfaces and air space inside commercial freeze dryers. For example, freeze dryers are inherently difficult to clean and sanitize and as a result, pose a risk for environmental contamination of food, vitamins and pharmaceuticals during the freeze drying process. The oxidative gases also help prevent the colonization of microorganisms inside equipment.

The system, method and apparatus may be applied intermittently during cleaning and sanitation done at regular or irregular intervals or continuously during operation of the freeze dryer.

The system, method and apparatus involves the installation of a REME generator on commercial freeze dryers. The installation may be external or internal. External installation was evaluated for the inactivation of microbiological contaminants in controlled environments. The effect of the REME system installed externally on inoculated stainless steel surfaces in an enclosed environment was determined in a controlled scientific study. Reductions in microbiological pathogens, including Listeria monocytogenes, E. coli O157:H7 and Salmonella were measured after 0, 2, 4, 8 and 24 hours exposure to the REME cell. The reductions ranged from 1.7 log/cfu cm2 after 2 hours of exposure to levels below the detection limit after 8 and 24 hours.

Still further advantages of the present invention will become apparent to those of ordinary skill in the art upon reading and understanding the following detailed description of the preferred embodiments.

### BRIEF DESCRIPTION OF THE DRAWINGS

The invention may take form in various components and arrangements of components, and in various steps and arrangements of steps. The drawings are only for purposes of illustrating preferred embodiments and are not to be construed as limiting the invention.

FIG. 1 is a schematic diagram of one embodiment of the system of the invention.

FIG. 2 is a schematic diagram of another embodiment of the system of the invention.

FIG. 3 is a schematic diagram of another embodiment of the system of the invention.

### DETAILED DESCRIPTION OF THE INVENTION

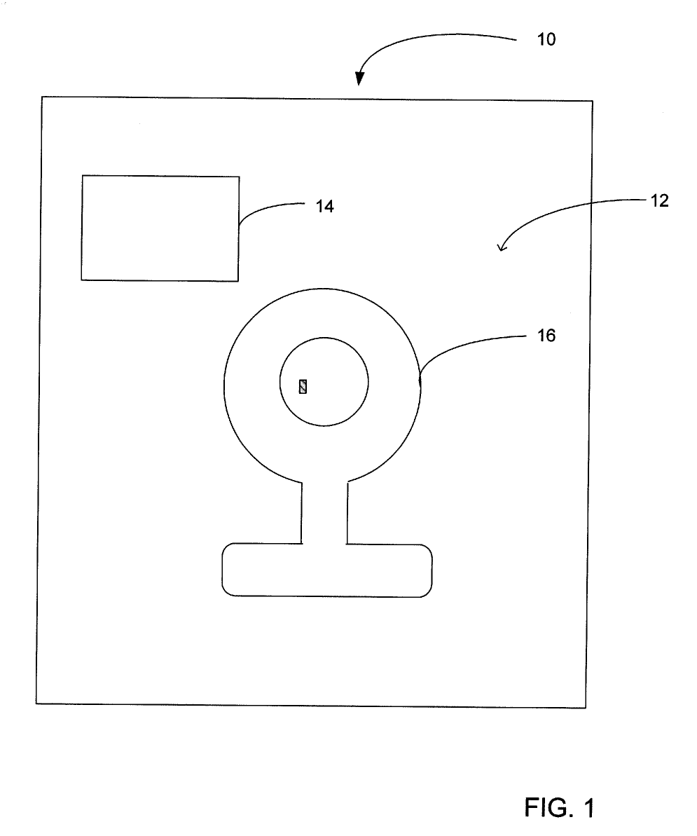

With reference to FIG. 1, The system of the invention is disclosed as element 10. A generally enclosed environment 12 is provided for the processing of food for humans and/or animals. In the environment 12 is provided an oxidative gas generator 14 such as one incorporating a Reflective Electro Magnetic Energy or REME system, such as that manufactured by RGF Environmental Group, Inc. Also included in the environment 12 is food processing equipment 16. Food processing equipment may be a commercial freeze dryer, but may also be any place where food is affected or altered. For example, food processing equipment includes equipment for chopping, freezing, cooking or otherwise preparing food for consumption.

REME or Reflective Electro Magnetic Energy utilizes an electro magnetic energy cell to create ionized hydro peroxides, an oxidizer plasma made from oxygen and humidity. The gas generator cell may incorporate a device that uses high voltage to ionize molecules of friendly oxidizers. REME can generate specifically charged ions (all positive or all negative) in the air, or it can create both polarities indiscriminately.

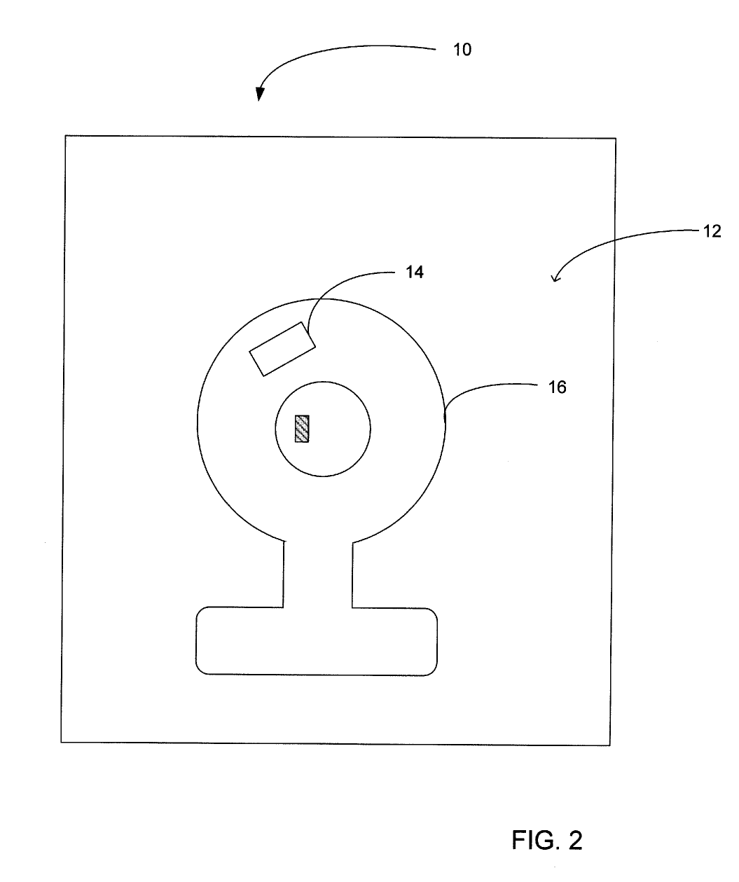

The oxidative gas generator 14 is installed in the environment 12 so that low levels of ozone, vaporized hydrogen peroxide and other oxidative gases are produced in proximity to the equipment. Equivalent generators of low levels of ozone, vaporized hydrogen peroxide and other oxidative gases as known in the art are also contemplated by this invention. The system, method and apparatus of the instant invention also contemplates the installation of an oxidative gas generator 14 on the surface of the equipment, as shown in FIG. 2 for a commercial freeze dryer 16.

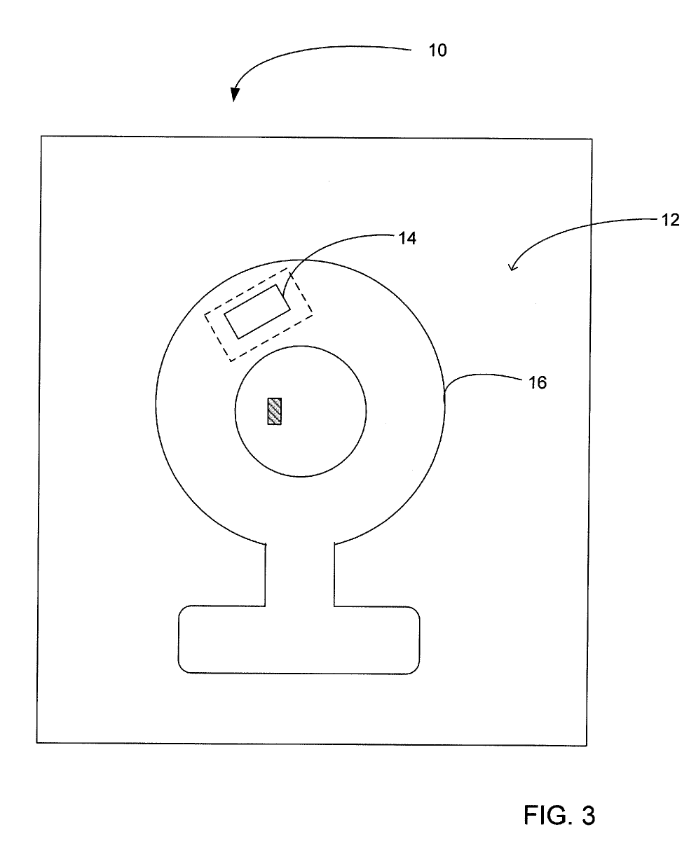

With reference to FIG. 3, the system of the invention is again shown as element 10. A generally enclosed environment 12 is provided for the processing of food. In this embodiment, an oxidative gas generator 14 is installed within the equipment. FIG. 2 shows the oxidative gas generator 14 located within the freeze drying area of a commercial freeze dryer 16.

External installation of an oxidative gas generator 14 was evaluated for the inactivation of microbiological contaminants in controlled environments. The effect of a REME system installed externally on inoculated stainless steel surfaces was determined in a controlled scientific study. Reductions in microbiological pathogens, including Listeria monocytogenes, E. coli O157:H7 and Salmonella in an enclosed environment were measured after 0, 2, 4, 8 and 24 hours exposure to oxidative gas generator 14. In this instance the oxidative gas generator was a REME cell. The reductions ranged from 1.7 log/cfu cm2 after 2 hours of exposure to levels below the detection limit after 8 and 24 hours.

In another embodiment of the invention, the oxidative gas generator would act as an adjunct to routine cleaning and sanitation. The combination of proper cleaning and sanitation and intermittent and/or continuous treatment with the oxidative gas generator 14 substantially reduce the risk of recontamination during food processing using the food processing equipment 16.

It will be appreciated by persons skilled in the art that the present invention is not limited to what has been particularly shown and described herein above. In addition, unless mention was made above to the contrary, it should be noted that the accompanying drawings are not to scale. A variety of modifications and variations are possible in light of the above teachings without departing from the scope and spirit of the invention.

What is claimed is:

1. A system for controlling microbial contamination, comprising:
a generally enclosed environment for the processing of food;
an oxidative gas generator located within the enclosed environment; and
food processing equipment within the generally enclosed environment;
wherein low levels of oxidative gases are produced in proximity to the food processing equipment.

2. The system of claim 1, wherein the oxidative gases include ozone and vaporized hydrogen peroxide.

3. The system of claim 1, wherein the oxidative gas generator is a REME cell.

4. The system of claim 1, wherein the generally enclosed environment includes a Cleaned in Place (CIP) system.

5. The system of claim 1, wherein the oxidative gas generator is installed on the surface of the food processing equipment.

6. The system of claim 1, wherein the food processing equipment comprises a commercial freeze dryer.

7. A device for controlling microbial contamination in a generally enclosed environment, comprising:
food processing equipment; and
an oxidative gas generator installed on the surface of the food processing equipment.

8. The device of claim 7, wherein the generally enclosed environment comprises a CIP system.

9. The device of claim 7, wherein the oxidative gas generator is a REME cell.

10. The device of claim 7, wherein the food processing equipment further comprises a commercial freeze dryer.

11. A method for controlling microbial contamination, comprising the steps of:
generally enclosing an environment;
locating an oxidative gas generator located within the enclosed environment; and
placing food processing equipment within the generally enclosed environment such that low levels of oxidative gases are produced in proximity to the food processing equipment.

12. The method of claim 11, wherein the generally enclosed environment comprises a CIP system.

13. The method of claim 11, further comprising the steps of:
placing food in the generally enclosed environment; and
processing of food in the generally enclosed environment.

14. The method of claim 11, wherein the food processing equipment comprises a commercial food dryer.

15. The method of claim 11, wherein the oxidative gas generator is located on the surface of the food processing equipment.

16. The method of claim 11, wherein the oxidative gas generator is a REME cell.
